(19) United States
(12) Patent Application Publication
(10) Pub. No.: US 2003/0008336 A1
(43) Pub. Date: Jan. 9, 2003

(54) Use of succinate as a diagnostic tool

(76) Inventors: Jack Edward Juni, Royal Oak, MI (US); Howard J. Saz, South Bend, IN (US); Becky S. Debruyn, Granger, IN (US)

Correspondence Address: BARNES & THORNBURG, 750-17TH STREET NW, SUITE 900, WASHINGTON, DC, 20006, US

(21) Appl. No.: 10/225,515

(22) Filed: Aug. 21, 2002

Related U.S. Application Data
(63) Continuation of application No. 09/623,036, filed on Aug. 24, 2000, now abandoned.
Application No. 09/623,036 is a 371 of international application No. PCT/US99/04011, filed on Feb. 24, 1999.
(60) Provisional application No. 60/075,596, filed on Feb. 24, 1998.

(51) Int. Cl. G01N 33/574; G01N 33/00; C12Q 1/26

(52) U.S. Cl. 435/7.23; 436/129

(57) ABSTRACT

A method and kit for detecting a disease state, characterized by a localized hypoxic/ischemic cell population or other cells with altered metabolisms similar to hypoxic/ischemic cells, in an animal is described. The method comprising the step of determining the concentration of succinate in a bodily fluid sample of said animal.

### CROSS-REFERENCE TO THE RELATED APPLICATIONS

This application is a continuation of U.S. patent application Ser. No. 09/623,036, filed Aug. 24, 2000, which is a U.S. national application of international application Ser. No. PCT/US99/04011, filed Feb. 24, 1998.

### BACKGROUND OF THE INVENTION

Many parasites that live in microaerophilic environments, such as the vertebrate intestine, survive in the absence of oxygen by generating energy through the reduction of fumarate by NADH to form succinate. Energy in the form of ATP is obtained as follows:

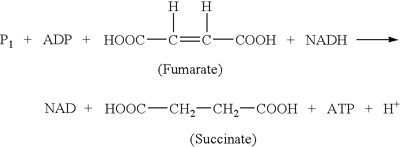

It has been found that other organisms will revert to a succinate accumulating metabolism to generate energy when exposed to hypoxic environments. Such organisms include numerous invertebrates (both parasitic and saprophytic), and vertebrates, including fish dwelling at the bottom of lakes and deep diving mammals such as whales and seals. In addition, it has been reported that succinate accumulates in localized regions of mammalian tissues that are subjected to hypoxic conditions. For example, it has been reported that mammalian rat heart in vivo and rat heart cells in culture, will accumulate succinate when the cells are deprived of oxygen.

The metabolism of humans and most other higher animals is highly dependent on the supply of adequate amounts of oxygen. Reduction in availability of oxygen may occur due to hypoxia or anoxia (reduced or absent oxygen concentration in the blood supplying a cell), to ischemia (reduced blood supply) or to a combination of ischemia and hypoxia. Such conditions result in a variety of metabolic changes and adaptations within the cell, some of which enhance the ability of the cell to produce energy despite reductions in available oxygen. Various tissues have differing tolerance to hypoxia and ischemia. If the resulting oxygen deprivation is sufficiently profound and prolonged, cells will die. In a “heart attack” or myocardial infarction, heart muscle cells die due to reduced blood flow (ischemia) caused by a blockage in the artery supplying those cells. In this setting, the heart cells receive insufficient quantities of oxygen and other nutrients to meet the basic demands for survival. In some patients, however, blood supply may be reduced only to the point that tissue function is impaired, but not to the point at which the cells die. Depending on the degree of ischemia, such conditions may exist transiently or may persist for extended periods. In the heart, this may result in reduced contractile function in ischemic tissues.

The response of myocardial tissue to hypoxic/ischemic conditions varies dependent on the severity and duration of the hypoxia/ischemia. When hypoxia/ischemia is severe and prolonged, myocyte cell death occurs and there is no recovery of contractile function of these cells. When hypoxia/ischemia is less severe myocytes may remain viable but exhibit depressed contractile function, which some suggest may be a protective mechanism whereby these cells attempt to reduce their oxygen demand.

Myocardial cells that remain viable but exhibit contractile dysfunction can be categorized into two groups (referred to generically as “jeopardized myocardium”). Myocardial tissue subjected to chronically low coronary blood flow exhibits a reversible decrease in the force of contraction, a phenomenon known as “hibernation”. A different sort of contractile dysfunction, “stunning” prevails following reperfusion after brief periods of ischemia. Accordingly, stunning typically involves contractile dysfunction after restoration of blood flow (perfusion), whereas myocardial hibernation typically involves contractile dysfunction during ongoing low myocardial blood flow (perfusion). Both types of contractile dysfunction are reversible with the appropriate therapy.

The identification of hibernating myocardium or myocardial stunning in patients is of considerable importance, since, upon revascularization, these conditions are generally reversible. Accordingly, screening patients that have poor myocardial function for the presence of hibernating myocardium or stunned myocardium allows for a more accurate prediction of the likelihood of clinical improvement following surgical intervention or other revascularization improvement therapies known to those skilled in the art (i.e., angioplasty, chemotherapeutics, etc.). Typically, a patient having less than 18% to 20% of the total left ventricle comprising hibernating myocardial tissue will have little or no improvement in heart function upon revascularization. However, patients having greater than 18% to 20% of the total left ventricle comprising hibernating myocardial tissue have a high likelihood for improvement in heart function following revascularization, and therefore are considered good candidates for successful surgical intervention or the application of other revascularization therapies.

Currently, positron emission tomography (PET) using a blood flow tracer and ¹⁸F-fluorodeoxyglucose (FDG) is often employed to identify hibernating and stunned myocardium and to triage patients for revascularization procedures such as bypass. This is a two-step process in which a blood flow scan of the heart is obtained to identify underperfused myocardium. A second scan is then obtained using FDG which is taken up by myocardial cells in proportion to cellular glucose uptake. Non-viable cells and scar tissue take up little or no FDG. Hibernating myocardium accumulates large amounts of FDG relative to the corresponding tissue perfusion. Stunned myocardium takes up FDG, but in small amounts relative to tissue perfusion. Thus, by comparison of the perfusion and FDG scans, heart tissue can be classified as normal, scar, hibernating or stunned. Both hibernating and stunned myocardium are considered “jeopardized”.

Although reduced blood oxygen content (hypoxia) is easily diagnosed using widely available techniques, the clinical diagnosis of tissue ischemia and infarction remains a vexing and important problem. In the heart, ischemia affects millions of people annually and represents the largest single cause of death in the United States. 5-7 million patients are seen annually in emergency departments in the U.S. for complaints of chest pain which may or may not be on the basis of cardiac ischemia. A number of enzyme-based blood tests (e.g. CPK isoenzymes) exist which are effective in diagnosing the presence of myocardial infarction (cell death due to ischemia). Unfortunately, many hours may be required for blood levels of these substances to become elevated after an infarction. This results in a costly delay in diagnosis and institution of treatment, frequently requiring patients to be admitted to the hospital for extended monitoring and blood sampling.

An even more vexing problem is the detection of ischemic events which do not result in actual infarction. Many patients experience transient episodes of cardiac ischemia which may or may not be associated with chest pain (angina). In patients presenting for emergency department evaluation with ischemia but who have not (yet) actually experienced cardiac cell death, diagnosis is notoriously difficult and inaccurate. Blood levels of cardiac enzymes are typically normal in such patients. Electrocardiogram (EKG) is negative or non-diagnostic in most patients. Nonetheless, a large number of such patients, especially those with so-called unstable angina, will progress to actual myocardial infarction, typically within the next few weeks after initial presentation to the emergency room. In an attempt to avoid sending such high-risk patients home, most emergency physicians have adopted a policy of admitting patients suspected of unstable angina to the hospital for observation and further testing. 70% of such patients are eventually proven to have no heart disease at all. This necessarily cautious practice results in excess health care costs of billions of dollars annually. Yet, despite this very careful pattern of medical practice, approximately 8% of all chest pain patients sent home from emergency departments go on to have a heart attack within the next four weeks. Such events represent a frequent cause of potentially preventable death from heart disease and constitute 25% of all malpractice settlements against emergency physicians.

Positron emission tomography, is the most accurate technique available in detecting the presence of hibernating myocardial tissues, but suffers the disadvantage that it costs $2,000 to $3,000 per test and is available in only a few centers. In addition the equipment required to conduct PET scans costs approximately 3-8 million dollars per facility. However, it has recently been reported that the use of even highly expensive means of assessing cardiac blood flow such as nuclear scanning of the heart (charges of $1,000 - $2,000 per test) resulted in a net reduction of cost to the health-care system for managing chest pain patients. Accordingly, a less expensive but accurate method of detecting cardiac ischemia, especially in the absence of acute infarction, will result in more appropriate management and treatment of these patients, will save lives and will reduce health- care costs. Thus, a diagnostic test for detecting viable ischemic cells, that is accurate and relatively inexpensive is desired.

Along with heart disease and stroke, cancer is a major cause of death and disability. Much cancer mortality is directly related to the difficulty in detecting the presence of a malignancy in a given patient. Current cancer screening methods are highly dependent on the detection of masses or lumps and on patient symptoms, e.g. pain. Unfortunately, these signs are absent in the early stages of most malignancies. Screening tests which look for masses (e.g. mammograms or chest x-rays) are notoriously inaccurate. In addition, they only screen certain portions of the body. It is impractical to x-ray the entire body to screen for cancer. Some blood tests for detecting cancer have been developed and a few (e.g. prostate specific antigen (PSA), carcinoembryonic antigen (CEA)) have reached clinical use. These blood tests, however, each test for only one or a few types of cancer. No existing blood test permits screening for a wide variety of cancers.

An additional problem in medical practice is the detection of cancer recurrence in patients who have already undergone some therapy. Tests such as x-ray or CT scan are particularly problematic in this setting. A blood marker applicable for a wide variety of tumor types would be of clinical benefit in monitoring patients for cancer recurrence and for guiding therapy.

The present invention is based on the surprising discovery that localized tissue of an animal subjected to hypoxic/ischemic conditions can be detected by measuring the concentration of succinate in an animal's peripheral blood and comparing the detected succinate levels to the basal succinate levels of the appropriate control group (i.e., whether the succinate levels exceed a “threshold” value). The technique claimed here permits an inexpensive, widely available diagnostic tool for identifying hypoxia/ischemia in patients at risk for, or suffering from, myocardial disease, a stroke, or cancer, and can be used to assess the likelihood of patient benefit from bypass surgery, angioplasty, pharmaceutical therapies or other therapies that restore or enhance blood and/or oxygen supply to the myocardial tissues.

In accordance with one embodiment, a diagnostic method for detecting a disease state, characterized by a localized hypoxic/ischemic cell population or other cells with altered metabolisms similar to hypoxic/ischemic cells in an animal, is described. Disease states that are characterized by a localized hypoxic/ischemic cell populations or other cells with altered metabolisms similar to hypoxic/ischemic cell populations include but are not limited to cancer (presence of hypoxic tumor cells), cardiovascular disease (presence of ischemic, hibernating or stunned myocardial cells) and stroke (presence of ischemic brain cells). The diagnostic method comprises the step of determining the concentration of succinate in a bodily fluid sample of said animal and optionally comparing the succinate concentration to the baseline succinate levels established for the applicable gender and age group. In one preferred embodiment the bodily fluid sample used to measure the succinate concentration is blood.

### SUMMARY OF THE INVENTION

The present invention is directed to measuring succinate levels in bodily fluids as a diagnostic indicator for the presence of localized ischemic tissue or regions of hypoxia that are indicative of the existence of jeopardized myocardium, the risk of stroke, or the presence of cancer cells. The method comprises the steps of obtaining a bodily fluid sample from the individual and determining if the concentration of succinate is greater than the clinical threshold value. This procedure can be used in conjunction with other standard techniques (such as PET) and can be used as a first screen to determine if additional diagnostic testing is warranted or the procedure can be used directly to determine treatment strategy.

### BRIEF DESCRIPTION OF THE DRAWINGS

FIGS. 1A-1F are graphic representations of succinate concentration verses time in the rat heart perfusion test. Rat hearts were excised from the rat, the blood was rinsed out of the heart and the heart was then hooked-up via the aorta to a perfusion apparatus which circulated a glucose-buffer solution through the heart in a predetermined O₂—N₂—CO₂ mixture at 37° C. as follows: FIG. 1A, gas mixture 70%O₂/ 25% N₂/ 5% CO₂; FIG. 1B, gas mixture 80%O₂/ 15% N₂/ 5% CO₂; FIG. 1C, gas mixture 95%O₂/ 5% CO₂; FIG. 1D, gas mixture 70%)O₂/ 25% N₂/ 5% CO₂; FIG. 1E, gas mixture 95%O₂/ 5% CO₂; FIG. 1F, gas mixture 95%O₂/ 5% CO₂.

FIGS. 2A and 2B are graphic representations of the data collected during the fumarate reduction assay. Perfused rat heart tissue was assayed anaerobically for fumarate reductase in mitochondrial preparations. Fumarate reductase was detected spectrophotometrically by measuring the anaerobic oxidation of NADH in the presence and absence of fumarate. Preliminary results from analysis of two separate homogenized rat myocardium samples (FIGS. 2A and 2B) indicate the presence of a fumarate reductase.

FIG. 3 is a graphic representation of the percent jeopardized myocardium. vs. the Ln [succinate] in whole blood samples obtained from human subjects. Approximately 70 samples of whole blood from patients and 15 samples of whole blood from volunteers (the controls) were analyzed for succinate concentration. Individuals were also analyzed by PET scanning for the presence of jeopardized myocardium

### DETAILED DESCRIPTION OF THE INVENTION

The present invention is directed to a novel diagnostic procedure for determining the presence of ischemic tissues or living tissues subject to hypoxic conditions in an animal or human. The method is based on the discovery that eukaryotic cells of an individual exposed to hypoxic and or ischemic conditions will release succinate, leading to detectable elevated levels of succinate in the bodily fluids of the animal. Accordingly, an increase in succinate levels over the basal levels of succinate found in healthy individuals is a diagnostic indicator that the individual is suffering from one or more diseases that are characterized by localized tissue ischemia, hypoxia or other similar metabolic derangements. Such diseases include, but are not limited to, myocardial disease, strokes, and carcinomas. Surprisingly, a localized region of hypoxia (i.e., such as ischemic myocardial tissue) can result in a significant increase in blood succinate levels.

The present invention utilizes the concentration of succinate in bodily fluids (including, but not limited to, whole blood, plasma, serum, urine and saliva) to detect the presence of cells that have an altered metabolism, caused by, or similar to that resulting from cells exposed to localized hypoxic or ischemic conditions. Measurements of succinate levels provide a simple, inexpensive screen for detecting myocardial hibernation and/or stunning, myocardial infarction, cerebral ischemia and/or infarction and ischemia/infarction of other body tissues. Furthermore, the concentration of succinate in the bodily fluids is directly correlated with the severity of these conditions. The diagnostic technique described in the present invention permits the detection of cells having an altered cellular metabolism and has potential for broad application in clinical medicine.

In one embodiment the method is used to determine the succinate levels in a warm blooded vertebrate species as a diagnostic tool for screening for diseases characterized by cells that exhibit altered metabolic functions. Three areas of clinical applicability are immediately evident: myocardial ischemia/infarct, cerebral ischemia/infarct and cancer.

Myocardial ischemia leads to heart failure, myocardial infarction and death. Current methods of detecting heart disease lack sensitivity and specificity. In the situation where an individual has severe ischemia and potentially has hibernating myocardium, current techniques for selecting patients for coronary artery bypass are imperfect. The best available technique, positron emission tomography, is reasonably accurate but costs $2,000 to $3,000 per test and is available in only a few centers. The technique described in the present invention permits inexpensive, widely available assessment of the likelihood of a patient benefiting from bypass surgery. For example, in individuals suffering from blockage or reduced blood flow to the myocardial tissues, scar tissue as well as hibernating and/or stunned tissue will be present. Hibernating tissue is tissue that is chronically ischemic, oxygen starved and is “struggling” to remain viable. Using standard analytical techniques it is difficult to distinguish scar tissue from hibernating tissue. However, once an adequate blood and oxygen supply is restored to hibernating tissues (i.e., by standard coronary artery bypass surgery) the hibernating tissue responds well and there is a high success rate with recovery of contractile function. However, the recovery rate after bypass surgery in patients without hibernating myocardial tissue is significantly lower. The presence of hibernating myocardial tissue can be detected by elevated succinate levels. Therefore the measurements of succinate levels in an individual can predict those patients that will benefit from bypass surgery or other revascularization therapies.

Cerebral ischemia and stroke are major causes of death and disability. The only successful therapy for stroke involves use of thrombolytic drugs, a high-risk procedure. Prior to administering therapy, a firm diagnosis must be established within 3 hours of stroke onset. There is currently no objective technique available for rapidly identifying and diagnosing brain ischemia. Such a simple tool might prove to be extremely important, particularly in emergency care patients who exhibit symptoms of a stroke. If within several hours of the attack, drugs, having the ability to lyse clots or having anti-clotting activity, are administered to the patient, the patient's life may be saved. However, if the stroke results from a hemorrhage rather than a blood clot, the patient may be placed in a more precarious state. By providing a simple, rapid and objective means of identifying brain ischemia/infarction, the technique disclosed herein may permit rapid and confident administration of thrombolytic therapy and more appropriate triage of patients towards or away from this high-risk procedure.

There is currently no available generalized blood test for cancer. All existing tests are specific to only a few tumor types, and may require biopsy for diagnosis. Testing for one of these types does not eliminate other tumor types. Accordingly, there is a need for a simple generalized assay to screen for the presence of tumor cells. It has also been reported that many, if not all solid tumors, lack adequate vascularization, and therefore have hypoxic regions. In general cancer cells have abnormal metabolisms that are similar to that seen in ischemic tissues even when the cancer cells are well oxygenated. As originally postulated by Otto Warburg in 1926, there is support that many, if not all, cancer cells metabolize anaerobically. Accordingly it is anticipated that many tumor cells will produce succinate as a by- product of their altered metabolism. Surprisingly, this localized production of succinate by solid tumors can be detected in the peripheral blood and used as an initial screen for tumors.

In accordance with one embodiment, the measurement of succinate levels in a vertebrate's bodily fluids can be used in accordance with the present invention for detecting the presence of cancer in a patient. The technique claimed here may prove useful in routine screening of patients for cancer, searching for tumor recurrence, and in assessing response of tumors to therapy. In accordance with one embodiment, patients can be screened for cancerous cells merely by means of determining succinate levels in peripheral blood.

It is well established that exercising muscle is also capable of functioning anaerobically (lactate production). Therefore, muscle diseases relating to metabolic defects may also produce high succinate levels and thus can be diagnosed by determining succinate levels in peripheral blood. This would constitute a broad and general method for determining any hypoxic/ischemic metabolism in human tissues.

Succinate is a common metabolite and methods for detecting and quantitating succinate levels have been described in the art and are familiar to those skilled in the art. One of the first methods for quantitative determination of succinic acid was a gravimetric procedure in which succinic acid was precipitated by silver ions and the precipitate was weighed. In a later improvement, gravimetry was replaced by titration of the excess silver ions.

Methods based on ion-exchange chromatography are more reliable than gravimetric assays. Lawson et al. (Acta (Wien) 1961, (pp.415-419)) were able to separate a large number of organic acids on a weak exchanger with formic acid as eluant. In addition, a relatively reliable method for detecting succinic acid was developed by Dimotaki-Kourakou (Ann. Falsif. Expert. Chim. 54, 70-83 (1961)). In this method, the organic acids of the sample are bound to a strongly alkaline ion-exchanger. After elution with an ammonium carbonate solution, the interfering organic acids are oxidized and the remaining succinate is titrated by silver nitrate/potassium rhodanide after extraction with ether. However, the method is very cumbersome, taking at least 15 hours to complete.

A gas-chromatographic method for detecting succinate in biological materials has also been developed (Chromatographia 12, 22-24 (1979)). Succinic acid in cerebrospinal fluid is methylated with a sulphuric acid/methanol mixture. The method takes at least 20 hours. The main disadvantage is that a large sample volume is needed for the assay.

A method has been described using high-performance liquid chromatography for determination of succinic acid in citrus fruits (J. Sci. Food Agric. 34, 1285-1288 (1983)). A preliminary separation of organic acids on ion-exchange columns is necessary to remove interfering substances. Chromatography on a propylamine column is very effective because of the relatively sharp peak that is obtained, but the level of detection for succinic acid is about 0.005%. The coefficient of variation is relatively poor in the lower concentration range.

The first enzymatic method for determination of succinic acid was suggested by Bemath et al. (Succinic Dehydrogenase, in: S. P. Colowick, N. O. Kaplan (eds.), Methods in Enzymology, Vol. V, Academic Press, New York 1962, pp. 597-614) succinic acid was converted to fumaric acid by means of the enzyme succinate dehydrogenase. Pires et al. (Z. Lebensm. Unterscuh. Forsch. 143, 96-99 (1970)) isolated a succinate dehydrogenase from Ascaris suum which oxidizes succinic acid to fumaric acid in the presence of tripotassium cyanoferrate (III). This enzyme is free from the enzymes of the citric acid cycle, and interference in the measurement due to the presence of other organic acids is therefore generally excluded.

The application of the enzyme succinyl-CoA synthetase (succinate thiokinase) for determining succinic acid is another enzymatic procedure used to quantitate succinate. Williamson et al. (Assays of Intermediates of the Citric Acid cycle and Related Compounds by Fluorometric Enzyme Methods, in: S. P. Colowick, N. O. Kaplan (eds.), Methods in Enzymology, vo. XIII, Academic Press, New York 1969, pp. 434-513) describe a procedure using succinyl-CoA synthetase from Escherichia coli. This enzyme converts succinic acid to activated succinate in the presence of ATP and CoA, to form ADP. ADP can be measured via the PK/LDH system using techniques known to those skilled in the art. The reliability of the assay depends strongly on the purity of the succinyl-CoA synthetase. The Michaelis constant of this enzyme toward succinate is relatively high (Kₘ ca. 10⁻³ mol/1), so that a relatively high activity has to be used for the assay. Since succinyl-CoA synthetase is often accompanied by a myokinase, interference is observed if the sample material contains ATP (tissues and biological materials). This problem can be avoided if the measurement of succinic acid is carried out using the GTP (ITP)-dependent enzyme succinyl-CoA synthetase from animal sources. A kit for detecting succinate via the succinyl-CoA method is commercially available from Boehringer Mannheim.

The present invention provides a method for detecting a disease state, characterized by a localized hypoxic/ischemic cell population or tissues comprising cells exhibiting metabolisms similar to those of hypoxic/ischemic cells. The method comprises the step of determining the concentration of succinate in a bodily fluid sample, such as blood. The method allows for a simple blood test that is capable of detecting the presence of tumor cells, ischemic brain cells, ischemic myocardial cells and hibernating/stunned myocardial cells and other ischemic/hypoxic tissues, or other cells with altered metabolisms similar to those states.

One embodiment of the present invention comprises a kit for use in detecting succinate levels in vertebrate bodily fluids. The kit comprises an enzyme that reacts with succinate to produce a detectable reactant, wherein the concentration of the reactant can be determined and can be correlated with the concentration of the succinate substrate. The detection scheme used to quantitate the reactant should be sensitive enough to determine the presence of succinate as low as 5 nmoles.

### EXAMPLE 1

### Rat Heart Perfusion Studies

Succinate levels in the perfusate were determined according to Kmetec and Bueding using Ascaris succinoxidase and coupling to the tetrazolium, dye, INT (Kmetec, Anal. Biochem., 16, 474 (1966)).

### Enzymatic Succinate Assay with Int

### Reagents

1. Succinate Std. -270 2 mg. 270.2 mg of disodium succinate was made to 10 ml (0.1M soln.) in water. Then, 0.1 ml of the 0.1M soln. was diluted to 100 ml (to produce a 0.1 mm solution) in water. For standard curve use 0.05, 0.1 and 0.2 ml of diluted soln.

2.0.2% Gelatin -20mg of Knox gelatin made to 10 ml in water.

3.0.2% INT- 20mg Sigma p-Iodonitrotetrazolium violet (also available from Pierce Biochemicals) made to 10 ml. in water.

Procedure

| Control Cuvette | Exptl. Cuvette |
|---|---|
| 0.1 ml. Tris buffer, | “ |
| 0.4 M (pH = 8.5) | |
| 0.2 ml gelatin | “ |
| 0.33 ml INT | “ |
| 0.33 ml H₂O | 0.33 ml, sample + |
| | H₂O |

←Zero Reading →

0.04 ml Ascaris succinate dehydrogenase is added to each Cuvette and the Cuvette incubated at 37° C. in dark until no further increase occurs at OD 540 mμ. Range=0.005-0.05 μmoles; 0.005μmoles reads about 0.060 after subtraction of blank (without succinate). Note: If ppt. forms upon addn. of INT to sample, the following procedure is employed: 0.05 ml sample+0.08 ml Tris (0.4M; pH 8,5)+1 ml 0.2% INT-Spin at 15,000 RPM for 30 min. Assay aliquot of supernatant for succinate. This procedure results in lower, but linear readings and should be adjusted accordingly.

### Preparation of Ascaris Succinoxidase

Preparation of Succinoxidase—Modified from Kmetec & Beuding, J. Biol. Chem. 236, 584 (1961).

### Reagents

a. 0.04M Tris, pH=8.5 containing 3.6 units of catalase per ml

b. 20% Sucrose+0.04 M Tris, pH 8.5

c. 1.8% Sodium deoxycholate

d. Saturated (NH₄)₂ SO₄

40 g. of fresh or frozen Ascaris muscle is minced and homogenized with 4 volumes (160 ml) of 0.04M Tris containing 3.6 units of catalase per ml. Homogenate was centrifuged at 6,000 ×g for 10 minutes. Enzyme remains in supernate, do not take fluffy layer. The supernate is then centrifuged at 100,000 ×g for 1.5 hours. Clear supernate was discarded. The residue was transferred with 2 volumes (80ml) of 20% sucrose-Tris solution. As much of the transparent glycogen layer on the bottom of the tube was left behind. It may be necessary to homogenize the particulate fraction to obtain a suspension. The suspension was recentrifuged at 144,000 ×g for 1.5 hours. Washed particles were resuspended in the original volume (40 ml) of sucrose-Tris and frozen overnight. The volume was adjusted so that 1 ml. of suspension was equivalent to 1 gram of muscle (40 ml). Actual volume was 43 ml. To this was added 8.5 ml of 1.8% Na deoxycholate slowly over a 15 minute period. If volume is different, deoxycholate added is adjusted accordingly. The mixture was incubated in the cold for two hours then frozen overnight. The volume is measured and saturated (NH₄)₂ SO₄ added slowly to 25% saturation. The mixture was then centrifuged at 10,000 RPM for 20 minutes and the supernatant was taken up to 55% saturation and spun at 10,000 RPM for 20 minutes. Residue of the 55% fraction was taken up in 18 ml of sucrose-Tris solution. The enzyme is stored frozen in test tubes with 0.3 ml per tube.

### Results

Rat hearts were excised from the rat, the blood was rinsed out of the heart and the heart was then hooked-up via the aorta to a perfusion apparatus which circulated a glucose-buffer solution through the heart in a predetermined O₂—N₂—CO₂ mixture at 37° C. Samples of the perfusate were collected, usually every 15 minutes. The hearts were maintained beating for varying time periods up to six hours. After finishing the collection of the last perfusate sample, heart was frozen and subsequently homogenized, centrifuged and assayed anaerobically for fumarate reduction in mitochondrial preparations. Fumarate reduction was detected spectrophotometrically by measuring the anaerobic oxidation of NADH in the presence and absence of fumarate (referred to as fumerate reductase).

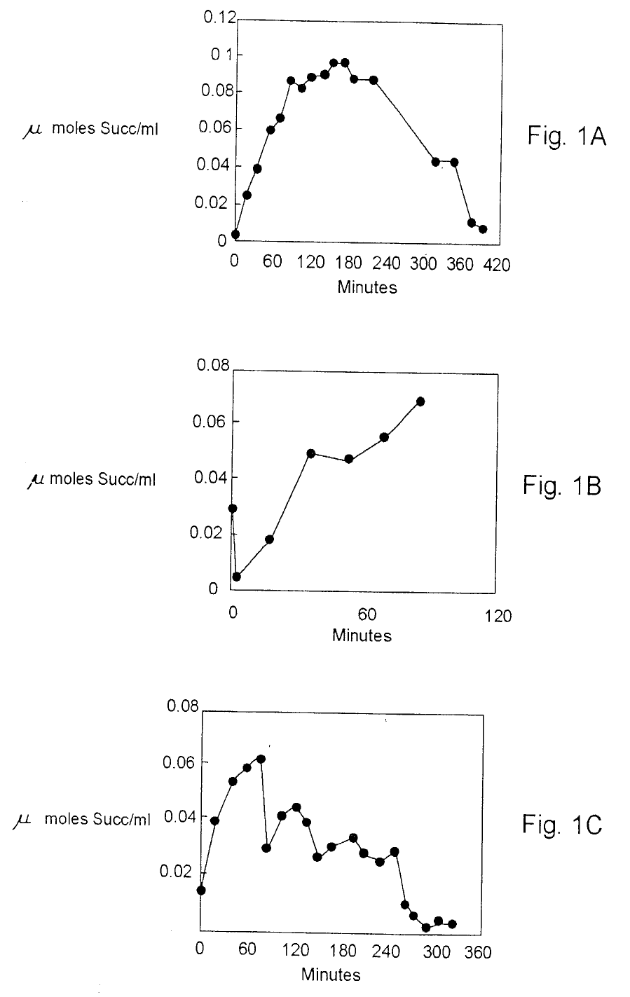

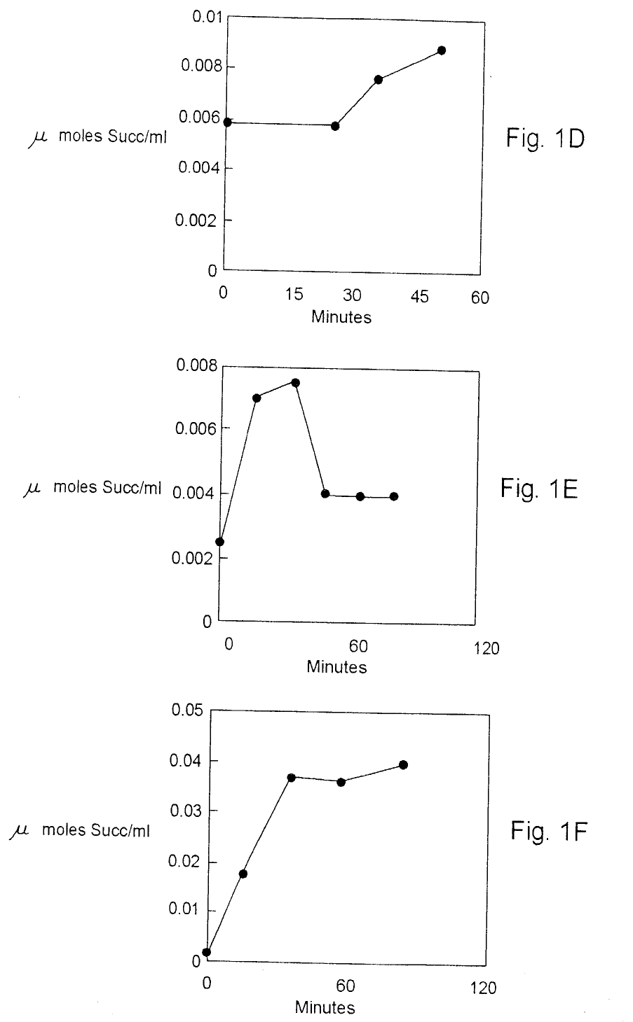

In all instances examined, succinate levels in the perfusate continued to increase until the hearts became erratic in their beats whereupon the succinate levels tend to decrease (See FIGS. 1A-1F). The gas mixtures utilized in the rat heart perfusion test comprise O₂ levels of 95% O₂, 80% O₂ and 70% O₂. Considerable amounts of succinate were formed at all of these O₂ levels. Lower concentrations of O₂ are expected to produce even higher concentrations of succinate and such conditions may be more physiologically relative since the usual concentration of O₂ in the lung would be about 20%. Although in some experiments there was an indication that small quantities of succinate were present at zero time, it took 10 to 15 minutes for the surgery and setting up. Therefore, it can not be determined whether or not there is at times a small pool of succinate present endogenously.

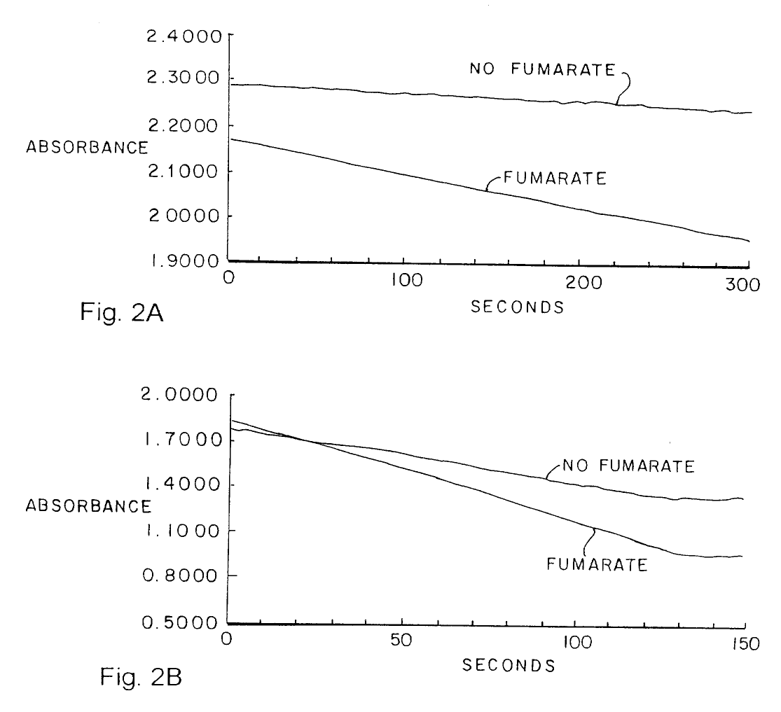

Preliminary results from analysis of the homogenized rat myocardium indicate the presence of a fumarate reductase. (See FIGS. 2A and 2B). Since oxygen can not be excluded completely, some NADH reductase activity could be present in the control. In addition, succinate was quantitated from heart mitochondria incubated anaerobically with fumarate plus NADH.

### EXAMPLE 2

### Screening Human Blood Samples for Succinate Levels

Numerous whole blood samples from patients have been analyzed for succinate including normal healthy “controls”. Approximately 70 samples of whole blood from patients and 15 samples of whole blood from volunteers (the controls) were analyzed for succinate concentration. Individuals were also analyzed by PET scanning for the presence of jeopardized myocardium after first obtaining peripheral blood samples. The blood samples of 22 patients were analyzed in blinded fashion for succinate level after the source patients' hearts had been PET scanned. After the succinate levels were determined the blood sample succinate levels were then correlated with the corresponding PET scan data and the percent of the left ventricle involved in hibernation or stunning jeopardized) was compared to the succinate blood levels.

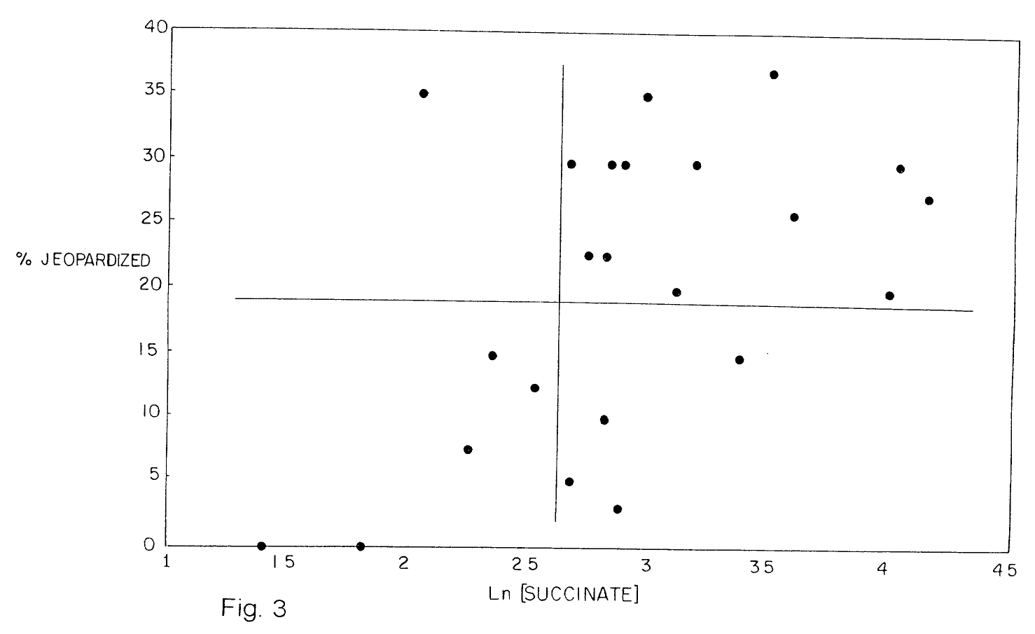

In summary, the concentrations of succinate in the blood of patients not only agreed well with the degree of myocardial hibernation, but also with another myocardial deficiency referred to as “stunning”. Plotting the percent jeopardized myocardium. vs. the Ln [succinate] gives a more easily interpreted curve (see FIG. 3). Clinically up to 18 percent jeopardized myocardium is considered as “insignificant” (noted by horizontal line in FIG. 3). A level of 15.2 nmoles of succinate/ml of peripheral blood was found to give the best separation between groups and was used as the “threshold” level of succinate. Individuals having succinate levels of less than 15.2 nmoles/ml were considered “negative” for purposes of this study (as noted by the vertical line in FIG. 3).

As demonstrated in FIG. 3, the sensitivity of blood succinate as an indicator of jeopardized myocardium (91%), as well as the specificity (86%) and accuracy (89%) place blood succinate levels as a viable diagnostic tool, at approximately the same level as PET scanning. All of the data obtained are in agreement with the results described in FIG. 3, and these findings agree well with the rat heart perfusion experiments described in Example 1.

To determine the “normal” blood levels of succinate in humans, the blood succinate levels of 15 “healthy” individuals were determined. The blood succinate levels ranged from 7.9 to 19.9 nmoles/ml in these individuals, with an average value of 15.2 nmoles/ml. The succinate levels in all “healthy” individuals were all very low in succinate, with 3 exceptions, all of whom were older persons. The higher levels of succinate in these individuals may be due to slower rates of blood flow to their hearts. Therefore it is anticipated that higher threshold values may need to be set for more elderly patients to account for higher overall succinate levels in those patients.

Numerous whole blood samples have been analyzed for succinate levels in addition to the samples that generated the data of FIG. 3, and the data generated from these additional samples were always consistent with each other and with the data of FIG. 3.

To determine the succinate blood levels, whole blood samples were first treated and decolorized in accordance with the following procedure:

### Extraction of Succinate from Whole Blood

Mix 1.0 ml blood* and 0.5ml 3.0 N H₂SO₄. Vortex and allow to stand for 10 min at room temperature. Spin for 20 min at 20,000 rpm.

Carefully remove supernatant and adjust pH to approx. 7.5 with 2.0 M KOH. Place the neutralized fraction on ice for 10 min. A precipitate forms. Spin for 20 min. at 20,000 rpm.

Remove the supernatant and add to it Norit A activated charcoal powder, 30 mg/ml of 2rd spin supernatant. Spin for 30 minutes at 20,000 rpm for the third time.

Carefully remove supernatant and again add Norit A, 30 mg/ml of 3rd spin supernatant. Spin again for 30 minutes at 20,000 rpm.

Remove the supernatant and assay for enzymatic succinate with INT, using 0.3 ml of final supernatant (Ref: Kmetec., Anal. Biochem. 16, 474 (1966)). Modification: Yee & Cohen, Anal. Biochem. 22, 530 (1968).

*Note: If whole blood is clotted, add 0.4 M EDTA, 0.02 ml/ml blood, and homogenize before adding H₂SO₄.

Two blood samples which were examined had very high succinate levels, but no apparent cardiac problem. Upon further investigation it was determined that one of the patients had lymphoma (a blood cancer). The second blood sample came from a woman whose breast cancer had been removed surgically one year earlier. The surgeon thought he had removed the whole tumor. PET scanning indicated a recurrence at the margin of the prior surgical resection. This finding was reinforced by the high concentrations of succinate found in the patient's post- operative blood, and subsequently by pathology. Therefore, it appears likely that succinate blood levels may increase with the occurrence of any tumor in the cells of the body, regardless of type or location. In accordance with one embodiment of the invention a simple blood test is used to screen for cancer. The test comprises determining the concentration of succinate in the sample to determine if the concentration is greater than the clinical threshold. This procedure constitutes a unique and excellent diagnostic tool.

1. A diagnostic testing method for detecting a disease state, characterized by a localized ischemic cell population in an animal, said method comprising the steps of:
selecting the animal, wherein the animal is not suffering from a globalized hypoxic condition,
measuring the concentration of succinate in peripheral blood of the animal; and
comparing the measured succinate concentration in the peripheral blood of the animal with a basal value for succinate concentration derived from measurements taken from a control group wherein hypoxic and/or ischemic cells are absent, to determine whether the succinate concentration in the peripheral blood of the animal is elevated, an elevated succinate concentration indicating the presence of said disease state.

2. The method of claim 1 wherein the localized cell population comprises tumor cells.

3. The method of claim 1 wherein the localized cell population comprises cancer cells.

4. The method of claim 1 wherein the localized cell population comprises ischemic brain cells.

5. The method of claim 1 wherein the localized cell population comprises ischemic or stunned myocardial cells.

6. The method of claim 1 wherein the localized cell population comprises hibernating myocardial cells.

7. The method of claim 1 wherein the animal is a human.

8. A diagnostic method for identifying individuals having localized ischemic tissues, said method comprising the steps of:
measuring the concentration of succinate in peripheral blood of said individuals;
comparing the measured succinate concentration in the peripheral blood of the individual with a basal value for succinate concentration derived from measurements taken from a control group wherein hypoxic and/or ischemic cells are absent;
determining whether the succinate concentration in the peripheral blood of each individual is elevated, an elevated succinate concentration indicating the presence of said ischemic tissues; and
identifying the individuals having the elevated succinate concentration as having localized ischemic tissues.

9. The method of claim 8 wherein the individual is a human.

10. The method of claim 9 wherein the human is not suffering from a globalized hypoxic condition.

11. The method of claim 9 wherein the human is not an older person and the basal level is about 15.2 nmoles/ml.

12. A diagnostic method for identifying a patient having localized ischemic tissues, said method comprising the steps of:
measuring the concentration of succinate in peripheral blood of said patient; and
comparing the measured succinate concentration in the peripheral blood of the patient with a basal value for succinate concentration derived from measurements taken from an appropriate control group wherein ischemic cells are absent, to determine whether the succinate concentration in the peripheral blood of the patient is elevated, an elevated succinate concentration indicating the presence of said ischemic tissues, and
wherein the patient is human and is not suffering from a globalized hypoxic condition.
